(19) United States
(12) Patent Application Publication
(10) Pub. No.: US 2023/0125482 A1
(43) Pub. Date: Apr. 27, 2023

(54) PROTEIN KINASE C MODULATORS

(71) Applicant: Young Therapeutics, LLC, Philadelphia, PA (US)

(72) Inventor: Lindon Young, Philadelphia, PA (US)

(21) Appl. No.: 17/914,147

(22) PCT Filed: Mar. 24, 2021
(86) PCT No.: PCT/US2021/023943

§ 371 Date: Sep. 23, 2022

Related U.S. Application Data
(60) Provisional application No. 62/994,098, filed on Mar. 24, 2020.
(60) Provisional application No. 63/017,488, filed on Apr. 29, 2020.

(51) Int. Cl. A01N 1/02; C07K 14/47

(52) U.S. Cl. CPC A01N 1/0226; C07K 14/4703; C07K 14/4705; C07K 2319/033; C07K 2319/10

(57) ABSTRACT

The present invention relates to compounds effective for the modulation of members of the protein kinase C (PKC) enzymatic family, including but not limited to, protein kinase epsilon (PKCε) inhibitors, protein kinase beta II (PKCβII) inhibitors, protein kinase zeta (PKCζ) inhibitors, and protein kinase C delta (PKCδ) activators. The present disclosure also relates to the mitigation of reperfusion (R) injury following the restoration of blood flow to previously ischemic (I) tissue. The present disclosure additionally relates to conjugates of PKC modulator peptides and a transduction domain of Trans-Activator of Transcription (TAT), and lipidated adducts thereof. The present disclosure further relates to a method of improving the therapeutic efficacy and solubility of compounds and drugs via dual conjugation of TAT and lipid moieties such as, for example, myristoyl.

### CLAIM OF PRIORITY

This application claims the priority of U.S. Provisional Patent Application Nos. 62/994,098, filed Mar. 24, 2020, and 63/017,488, filed Apr. 29, 2020, which are incorporated herein by reference.

### SEQUENCE LISTING

The instant application contains a Sequence Listing which has been submitted electronically in ASCII format and is hereby incorporated by reference in its entirety. The ASCII copy, created on Mar. 24, 2021, is named 1620_001_PCT_SL.txt and is 7,432 bytes in size.

### BACKGROUND

There are 800,000 heart attack patients per year in the US, and virtually all the patients who undergo coronary angioplasty or bypass surgery will be candidates for a therapy that prevents ischemia/reperfusion injury (IRI). IRI is a phenomenon that occurs when oxygenated blood is reintroduced into ischemic tissue. For example, when a cardiac surgeon re-opens the clogged coronary artery (coronary angioplasty) that caused the heart attack, the re-introduction of blood can cause further additional damage. Following ischemia, it is essential that oxygen-carrying blood flow is restored (reperfusion) to the ischemic area. However, the well-known phenomenon associated with reperfusion that results in cytokine activation and the formation of reactive oxygen species (ROS) leads to additional cell damage and death (i.e., IRI). To date, no treatment has been able to expeditiously (within minutes) prevent the generation of ROS that occurs following reperfusion.

Additionally, in organ transplantation, success is often limited due to ischemic/reperfusion injury. Isolated human hearts deprived of oxygen for more than four hours progressively loose vigor and often do not survive in recipient hosts. Other organs such as the kidney, liver, pancreas and lung are also subjected to tissue and cellular damage when removed from their hosts prior to transplantation. This damage is due to hypoxic conditions and a lack of circulation, which normally delivers physiological concentrations of oxygen and nutrients, and removes toxic compounds produced by an organs cells. Organ transplants have a higher frequency of success when performed immediately after excision from their hosts.

Restoration of blood flow is the primary objective for treatment of organ tissue experiencing prolonged ischemia, e.g., during transplant and after a heart attack. However, reperfusion of blood flow induces endothelium and myocyte injury, resulting in organ dysfunction. The sequential events associated with reperfusion injury are initiated by endothelial dysfunction which is characterized by a reduction of the basal endothelial cell release of nitric oxide (NO) within the first 2.5-5 min post-reperfusion. The decrease in endothelial derived NO is associated with adhesion molecule up-regulation on endothelial and polymorphonuclear (PMN) leukocyte cell membranes. This event promotes PMN/endothelial interaction, which occurs by 10 to 20 min post-reperfusion, and subsequent PMN infiltration into the myocardium is observed by 30 min post reperfusion.

Chemotactic substances released from reperfused tissue and plasma factors activate PMNs that augment PMN release of cytotoxic Substances (i.e. Superoxide anion) and contribute to organ dysfunction following ischemia/reperfusion. Superoxide combines with NO to produce peroxynitrite anion thus reducing the bioavailability of NO and promotes endothelial dysfunction and PMN infiltration after myocardial ischemialreperfusion.

The inventors own prior patent applications, U.S. Patent Application Publication Nos. 2006/0160062, 2007/0148628, 2008/0311553, 2012/0141973, 2014/0220549, and 2016/0302406 disclose the use of protein kinase epsilon (PKCε) inhibitors, protein kinase beta II (PKCβII) inhibitors, protein kinase zeta (PKCζ) inhibitors, and protein kinase C delta (PKCδ) activators to ameliorate ischemia injury in organs.

Therefore, there remains a need for a composition of improved quality that can protect the organ from reperfusion injury after ischemia, so that the organ can resume proper function after restoration of blood flow.

### SUMMARY OF THE INVENTION

An aspect of the present invention relates to compounds effective for the modulation of members of the protein kinase C (PKC) enzymatic family, including but not limited to, protein kinase epsilon (PKCε) inhibitors, protein kinase beta II (PKCβII) inhibitors, protein kinase zeta (PKCζ) inhibitors, and protein kinase C delta (PKCδ) activators. The present disclosure also relates to the mitigation of reperfusion (R) injury following the restoration of blood flow to previously ischemic (I) tissue. The present disclosure additionally relates to conjugates of PKC modulator peptides and a transduction domain of Trans-Activator of Transcription (TAT), and lipidated adducts thereof. The present disclosure further relates to a method of improving the therapeutic efficacy of compounds and drugs via dual conjugation of TAT or other positive charged peptide carriers (e.g. polyarginines) and lipid moieties such as, for example, myristoyl. The combination of Myr+TAT combined with the PKC modulators described herein results in an unexpected increase in potency (up to 10,000× to reduce infarct size; FIG. 3) and solubility, and suggests that the potency of any intracellular target peptide or small molecule cargo would be dramatically increased when combined with Myr−TAT and is the basis of this invention.

Preferably, the compound of the present invention is a peptide have the general structure of Formula I

Myr−TAT−L−P (Formula I)

wherein Myr is myristoyl; TAT is the transduction domain of Trans-Activator of Transcription (TAT) or lipidated adducts thereof; L is a linker peptide; and P is a PKCε inhibitor peptide (PKCε−), PKCβII inhibitor peptide (PKCβII−), PKCζ inhibitor peptide (PKCζ−), or PKCδ activator peptide (PKCβ+).

A further aspect of the present invention includes methods of using the compounds of the present invention. These include methods for preserving an organ for transplantation, for protecting an ischemic organ from damage, for attenuating organ dysfunction after ischemia, and for protecting an organ from damage when isolated from the circulatory system.

Other aspects of the invention, including apparatuses, devices, kits, processes, and the like which constitute part of the invention, will become more apparent upon reading the following detailed description of the exemplary embodiments.

### BRIEF DESCRIPTION OF THE FIGURES

FIG. 1 is a representative depiction of an ex vivo heart provided in a Langendorff apparatus as used in EXAMPLE 1.

FIG. 2 depicts representative tracings of the maximal rise of left ventricular developed pressure (LVDP) [+dp/dtₘₐₓ; mmHg/s] and the maximal rate of decline of LVDP [−dp/dtₘᵢₙ; mm/Hg/s] as measured after 50 minutes of reperfusion in ischemic ex vivo rat hearts subjected to 30 minutes of ischemia that were either treated with vehicle (left) or 100 nM Myr+TAT+PKCε− for the first five minutes of reperfusion.

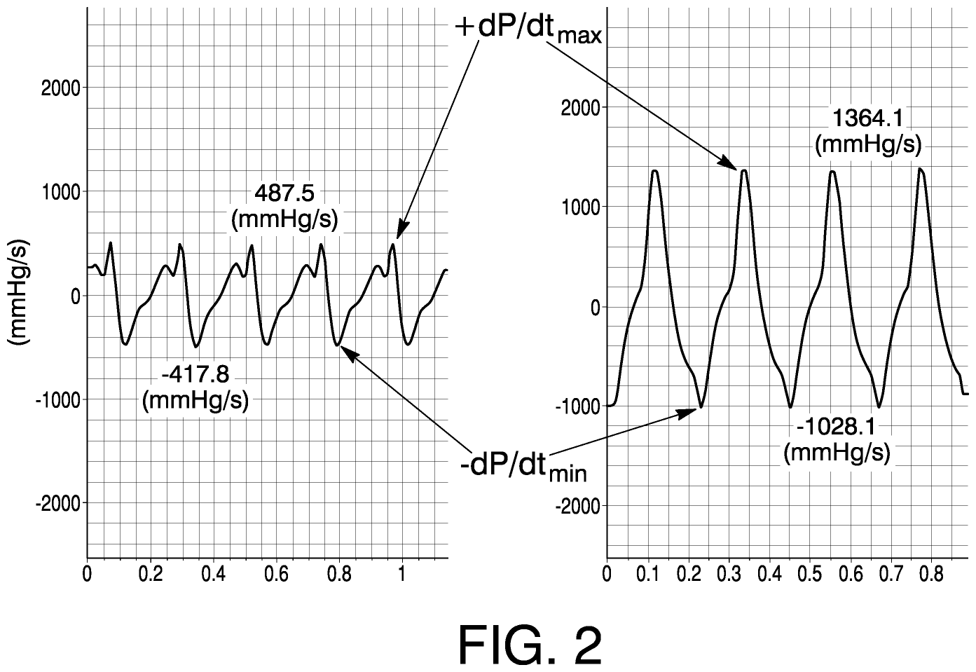

FIG. 3 is a chart summarizing the mean infarct size in ex vivo rat hearts subjected to 30 minutes of ischemia and 50 minutes reperfusion. Hearts were treated with selected PKC epsilon inhibitor compounds for the first five minutes of reperfusion.

FIG. 4 is a chart summarizing the mean infarct size in ex vivo rat hearts subjected to 30 minutes of ischemia and 50 minutes reperfusion. Hearts were treated with selected PKC beta II modulating compounds for the first five minutes of reperfusion.

FIG. 5 is a chart summarizing maximal rate of rise of left ventricle developed pressure (dP/dTₘₐₓ) 50 minutes after reperfusion and treatment of ischemic rat hearts with a myristoylated PKCε inhibitor or control compounds that contain a scrambled PKC modulator sequence, lack a TAT sequence, or are not myristoylated.

FIG. 6 is a chart summarizing the time course of effects of PKCε inhibitors on maximal rate of rise of left ventricle developed pressure (dP/dTₘₐₓ) in rat hearts undergoing 50 minutes of reperfusion after 30 minutes of ischemia.

FIG. 7 is a chart summarizing the effects PKCβII inhibitors, PKCβII scrambled control inhibitor peptide (PKC βII-scram), PKCβII activators, and superoxide dismutase (SOD) on phorbol myristate acetate (PMA)-stimulated superoxide (SO) release in rat polymorphonuclear leukocytes (PMNs) expressed as change in ferrocytochrome c absorbance at 550 nm (*P<0.05 is Myr−PKCβII− vs. PMA and **P<0.01 Myr−Tat−PKCβII− vs. all groups except SOD or Myr−TAT−PKCβII−; ++P<0.01 Myr−PKCβII− scram. vs. all groups).

FIG. 8 is a chart depicting the time course of different concentrations of Myr−TAT−PKCβII inhibitor effects on PMA-stimulated superoxide release in rat polymorphonuclear leukocytes expressed as change in ferrocytochrome c absorbance at 550 nm.

FIG. 9 is a chart depicting the time course of Myr−TAT−PKCβII inhibitor effects on PMA-stimulated superoxide release in rat polymorphi-muclear leukocytes as compared with control compounds and PKCβII inhibitors lacking a TAT induction sequence.

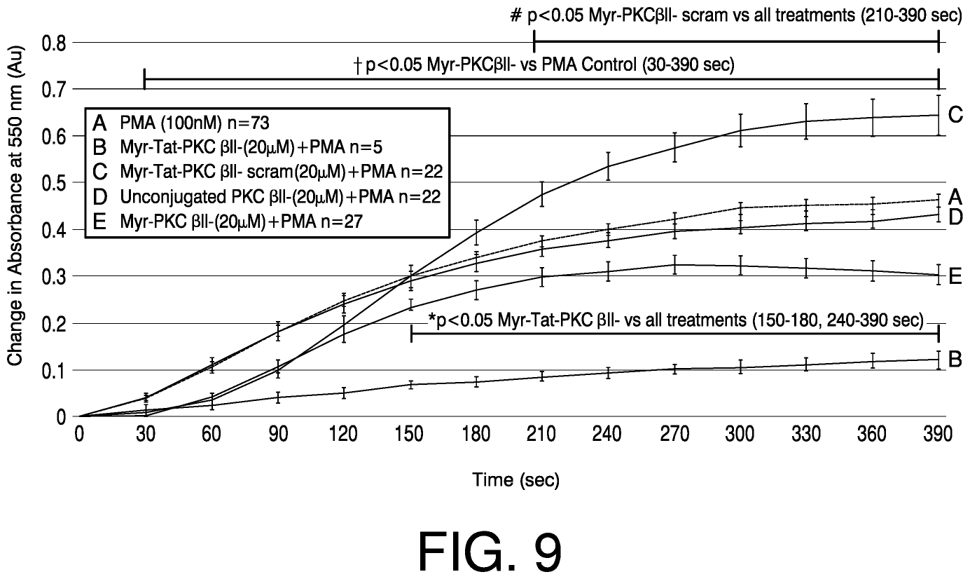

### DETAILED DESCRIPTION

This application relates to compounds effective for the modulation of members of the protein kinase C (PKC) enzymatic family, including, without limitation, protein kinase epsilon (PKCε) inhibitors, protein kinase beta II (PKCβII) inhibitors, protein kinase zeta (PKCζ) inhibitors, and protein kinase C delta (PKCδ) activators. The present disclosure also relates to the mitigation of reperfusion (R) injury following the restoration of blood flow to previously ischemic (I) tissue. The present disclosure additionally relates to conjugates of PKC modulator peptides and a transduction domain of Trans-Activator of Transcription (TAT), and lipidated adducts thereof. The present disclosure further relates to a method of improving the therapeutic efficacy of compounds and drugs via dual conjugation of TAT or other positive charged peptide carriers (e.g. poly arginines) and lipid moieties such as, for example, myristoyl.

Preferably, the compound of the present invention is a peptide have the general structure of Formula I

Myr−TAT−L−P (Formula I)

wherein Myr is myristoyl; TAT is the transduction domain of Trans-Activator of Transcription (TAT) or lipidated adducts thereof; L is a linker peptide; and P is a modulator of PKCε, preferably PKCε inhibitor peptide, PKCβII inhibitor peptide, PKCζ inhibitor peptide, PKCδ activator peptide, or combinations thereof. The myristoyl group is preferably an N-myristolation. TAT preferably has the amino acid sequence YGRKKRRQRRR (SEQ ID NO: 1), GRKKRRQRRR (SEQ ID NO: 2), or RKKRRQRRR (SEQ ID NO: 3). The PKCε inhibitor peptide preferably has the amino acid sequence EAVSLKPT (SEQ ID NO: 4). The PKCβII inhibitor peptide preferably has the amino acid sequence SLNPEWNET (SEQ ID NO: 5). The PKCδ activator peptide preferably has the amino acid sequence MRAAEDPM (SEQ ID NO: 6). The PKCζ inhibitor peptide preferably has the amino acid sequence SIYRRGARRWRKL (SEQ ID NO: 7) or SIYRRGARRWRKLYRAN (SEQ ID NO: 8). The linker sequence is a peptide, preferably having four amino acids or less, more preferably two amino acids. An exemplary linker can have the following amino acid sequence of CC, GG, CCC, GGG, CGC, GCG, CCCC (SEQ ID NO: 9), GGGG (SEQ ID NO: 10), CGGC (SEQ ID NO: 11), GCCG (SEQ ID NO: 12), CGCG (SEQ ID NO: 13), or GCGC (SEQ ID NO: 14). The preferred amino acid sequence for the linker is CC or GG.

### Protein Kinase C

The benefit of reperfusion for patients who suffer myocardial ischemia can be constrained by reperfusion-induced cardiac contractile dysfunction and myocardial cell injury. Reperfusion injury is characterized by an increase in oxygen-derived free radicals that damage cell membrane permeability leading to intracellular calcium overload and cardiac hypercontracture. A principal reactive oxygen species is superoxide anion (SO), which is produced by several sources that include polymorphonuclear leukocyte (PMN) and endothelial NADPH oxidase, incomplete oxidative phosphorylation in mitochondria, and endothelial nitric oxide (NO) synthase (eNOS) when essential eNOS cofactor, tetrahydrobiopterin (THB), is oxidized to dihydrobiopterin (DHB). Superoxide is further converted to hydrogen peroxide (H₂O₂) by superoxide dismutase, then H₂O₂ is converted to water by catalase. However, these endogenous oxidative stress defense mechanisms can be overwhelmed during reperfusion. Superoxide can also attenuate the bioavailability of NO through the formation of peroxynitrite anion. An abrupt decrease in endothelium-derived NO can occur within 5 minutes of reperfusion and results in endothelial dysfunction. Endothelial dysfunction an promote the upregulation of endothelial adhesion molecules [e.g., intracellular adhesion molecule-1 (ICAM-1)] to facilitate PMN adherence and infiltration. After 30 minutes, the transmigrated PMNs can release cytotoxic substances such as superoxide radicals to directly injure the myocardium that contributes to cardiac contractile dysfunction.

Protein kinase C (PKC) is a family of protein kinase enzymes involved in myocardial ischemic/reperfusion (MI/R). PKC regulates eNOS in both roan and rats and can exert positive or negative regulation of nitric oxide (NO) release with respect to its different isoforms. Inhibition of PKC beta II (βII), PKC delta (δ), and PKC zeta (ζ) can increase NO release from nonischemic rat aortic segments, while activation of PKC epsilon (ε) can lead to eNOS phosphorylation and increased expression and NO release.

Different PKC isoforms can also regulate Intercellular Adhesion Molecule 1 (ICAM-1) expression under different biological conditions. For instance, PKCβ inhibition can attenuate ICAM-1 expression in the kidney of diabetic rats and improves renal function. Conversely, activation of PKCζ by tumor necrosis factor-alpha can increase polymorphonuclear leukocyte (PMN) adhesion to human pulmonary artery endothelial cells through phosphorylation of ICAM-1. In addition, upregulation of PKCε accompanying with enhanced ICAM-1 expression are observed in salt-sensitive hypertensive rats. In PMNs, broad-spectrum PKC activation using PMA can increase superoxide (SO) release via phosphorylation of cytosolic factor p47phox that is required for NADPH oxidase activation. Whereas, selective PKCδ activation can reduce the PMA-induced PMN SO release.

### Compounds of the Disclosure

Selected compounds of the disclosure are peptides provided in TABLE 1, wherein CC represents two adjacent cysteine residues being used the linker (L in Formula 1), and Myr is myristoyl. N-myristolylated TAT induction peptides (e.g. Myr-YGRKKRRQRRR (SEQ ID NO: 1 with N-myristoylation)) in selected compounds can facilitate membrane translocation of the payload PKC-modulating peptide, and thus promote interaction of the PKC-modulating peptide with the target intracellular substrate (e.g. receptor for activated C kinase (RACK)). Under certain conditions, the CC linker may contain side chains that are bound to each other by a disulfide bond (i.e. intrachain disulfide bond). Myr+TAT and the PKC-modulating peptides car be linked by moieties other than CC, such as a glycyl-glycine linker (GG), or an interchain disulfide bond between a PKC modulating peptide and Myr+TAT. When interchain disulfide bonds, where two separate peptide chains are conjugated to one another solely by a disulfide bond between cysteine side chains of each peptide chain, can be reducible by the intracellular environment and facilitate liberation of unconjugated payload sequences (e.g. the PKCε inhibitor peptide, PKCβII inhibitor peptide, PKCζ inhibitor peptide, or PKCδ activator peptide) within the cytosol after membrane translocation. A peptide bond within the disulfide bridge linker may be added to improve the purity of synthesis if needed. Scrambled Myr+PKCε−, scrambled Myr+TAT PKCε−, scrambled Myr+TAT+PKCβII−, and scrambled Myr+TAT+PKCδ+ represent additional controls for the Myr and Myr−TAT induction moieties and the payload sequences. Herein, “−” is used to indicate an inhibitor and “+” is used to denote an activator. For example, PKCε−, PKCβII−, and PKCζ− denote PKCε inhibitor, PKCβII inhibitor, and PKCζ inhibitor, respectively; and PKCδ+ denotes PKCδ activator.

TABLE 1

| Compound | | | | SEQ |
|---|---|---|---|---|
| abbrevi- | | Mol. | Descrip- | ID |
| ation³ | Structure¹ | Weight | tion | NO:² |
| Myr + | Myr- | 3045 | Protein | 15 |
| TAT + | YGRKKRRQRRRC | | Kinase C | |
| PKCβII- | CSLNPEWNET | | betaII | |
| | | | inhibitor | |
| Myr + | Myr- | 3045 | Scrambled | 16 |
| TAT + | YGRKKRRQRRRC | | Protein | |
| PKCβII- | CWNPESLNTE | | Kinase C | |
| scrambled | | | betaII | |
| | | | inhibitor | |
| Myr + | Myr-SLNPEWNET | 1300 | Protein | 5 |
| PKCβII- | | | kinase C | |
| | | | betaII | |
| | | | inhibitor | |
| Myr + | Myr-WNPESLNTE | 1300 | Scrambled | 27 |
| PKCβII- | | | Protein | |
| scrambled | | | kinase C | |
| | | | betaII | |
| | | | inhibitor | |
| PKCβII- | SLNPEWNET | 1090 | Protein | 5* |
| | | | kinase C | |
| | | | betaII | |
| | | | inhibitor | |
| | | | (Unconju- | |
| | | | gated) | |
| Myr + | Myr- | 2800 | Protein | 18 |
| TAT + | YGRKKRRQRRRC | | Kinase C | |
| PKCϵ- | CEAVSLKPT | | epsilon | |
| | | | inhibitor | |
| Myr + | Myr- | 2800 | Scrambled | 19 |
| TAT + | YGRKKRRQRRRC | | Protein | |
| PKCϵ- | CLSETKPAV | | Kinase C | |
| scrambled | | | epsilon | |
| | | | inhibitor | |
| TAT + | YGRKKRRQRRRC | 2590 | Protein | 18* |
| PKCϵ- | CEAVSLKPT | | Kinase C | |
| | | | epsilon | |
| | | | inhibitor | |
| Myr + | Myr-EAVSLKPT | 1054 | Protein | 4 |
| PKCϵ- | | | Kinase C | |
| | | | epsilon | |
| | | | inhibitor | |
| Myr + | Myr-LSETKPAV | 1054 | Scrambled | 20 |
| PKCϵ- | | | Protein | |
| scrambled | | | Kinase C | |
| | | | epsilon | |
| | | | inhibitor | |
| Myr + | Myr- | 2875 | Protein | 21 |
| TAT + | YGRKKRRQRRRC | | kinase C | |
| PKCδ+ | CMRAAEDPM | | delta | |
| | | | activator | |
| Myr + | Myr- | 2875 | Scrambled | 22 |
| TAT + | YGRKKRRQRRRC | | Protein | |
| PKCδ+ | CAMEADPMR | | Kinase C | |
| scrambled | | | delta | |
| | | | activator | |
| Myr + | Myr-SVEIWD | 958 | Protein | 23 |
| PKCβII+ | | | kinase C | |
| | | | betaII | |
| | | | activator | |
| Myr- | Myr-MRAAEDPM | 1130 | Protein | 6 |
| PKCδ+ | | | kinase C | |
| | | | delta | |
| | | | activator | |
| Myr + | Myr- | 3573 | Protein | 24 |
| TAT + | YGRKKRRQRRRCC | | kinase C | |
| PKCζ- | SIYRRGARRWRKL | | zeta | |
| version 1 | | | inhibitor | |
| | | | version 1 | |
| Myr + | Myr- | 4249 | Protein | 25 |
| TAT + | YGRKKRRQRR | | kinase C | |
| PKCζ- | RCCSIYRRGA | | zeta | |
| version 2 | RRWRKLYRAN | | inhibitor | |
| | | | version 2 | |
| Myr + | Myr-SIYRRGA | 1928 | Protein | 7 |
| PKCζ- | RRWRKL | | kinase C | |
| version 1 | | | zeta | |
| | | | inhibitor | |
| | | | version 1 | |
| Myr + | Myr- | 2504 | Protein | 8 |
| PKCζ- | SIYRRGARRWR | | kinase C | |
| version 2 | KLYRAN | | zeta | |
| | | | inhibitor | |
| | | | version 2 | |
| Myr + | Myr- | 3673 | Scrambled | 32 |
| TAT + | YGRKKRRQRRRCC | | kinase C | |
| PKCζ- | RLYRKRIWRSAGR | | zeta | |
| version 1 | | | inhibitor | |
| scrambled | | | version 1 | |
| Myr + | Myr- | 4249 | Scrambled | 33 |
| TAT + | YGRKKRRQRRRC | | kinase C | |
| PKCζ- | CRLRYR | | zeta | |
| version 2 | NKRIWRSAYAGR | | inhibitor | |
| scrambled | | | version 2 | |

¹Myr denotes N-terminal myristoylation.
²N-terminal myristoylation is added to the sequence unless otherwise indicated.
³When linker is present, CC is used.
*No myristoylation.

In some embodiments, amino acid sequence variants of the sequences described herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the compound. Amino acid sequence variants of a compound described herein may be prepared by introducing appropriate modifications into the nucleotide sequence encoding the compound, or by peptide synthesis. Such modifications include, for example, deletions from, and/or insertions into and/or substitutions of residues within the amino acid sequences of the compound. Any combination of deletion, insertion, and substitution can be made to arrive at the final construct, provided that the final construct possesses the desired characteristics, e.g., mitigation of I/R injury.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for embodiment, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

The peptides disclosed herein (including functional portions and functional variants thereof) can comprise synthetic amino acids in place of one or more naturally-occurring amino acids. Such synthetic amino acids can include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenvialanine, 4-chlorophenylalanine, 4-carboxyphenytalanine, β-phenylserine β-hydroxyphenylalanine, phenylglycine, α-naphthylalanme, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, hornophenylalanine, and α-tert-butylglycine.

The peptides disclosed herein can additionally comprise non-proteogenic acids in place of one or more proteogenic amino acids amino acids. Such non-proteogenic acids can include, for example, β-alanine, cystine, cystathionine, lanthionine, t-leucine, norleucine, homonorleucine, omithine, allothreonine, homocysteine, homoserine, isovaline, norvaline, sarcosine, N-ethyl glycine, N-propyl glycine, N-isopropyl glycine, N-methyl alanine, N-ethyl alanine, N-methyl β-alanine, N-ethyl β-alanine, and isoserine.

The present disclosure further contemplates the peptides described herein can be associated with modifications of one or more amino acids of the polypeptide constructs. Non-limiting examples of modifications include phosphorylation, acylation including acetylation and formylation, glycosylation (including N-linked and O-linked), amidation, hydroxylation, alkylation including methylation and ethylation, ubiquitination, addition of pyrrolidone carboxylic acid, forrnation of disulfide bridges, sulfation, myristoylation, palmitoylation, isoprenylation, famesylation, geranylation, glypiation, lipoylation and iodination.

### Administration

A pharmaceutical composition containing a peptide can be administered to patients along with pharmaceutical excipients or diluents. Non-limiting examples of suitable pharmaceutical excipients or diluents include starch, glucose, lactose, sucrose, gelatin, malt, rice, flour, chalk, silica gel, magnesium carbonate, magnesium stearate, sodium stearate, glycerol monostearate, talc, sodium chloride, dried skim milk, glycerol, propylene, glycol, water, ethanol, buffered water, and phosphate buffered saline. These compositions can take the form of drops, solutions, suspensions, tablets, pills, capsules, powders, and sustained-release formulations. In some embodiments, the composition is an eardrop. In another embodiment the composition containing a peptide in any form could be further modulated using suitable excipients and diluents including lactose, dextrose, sucrose, sorbitol, mannitol, starches, gum acacia, calcitun phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents. The compositions can be formulated in a unit dosage form, each dosage containing, for example, from about 1 ng to 1000 mg of the peptide. In some embodiments, a dose contains from 100-1000 mg of the peptide. In some embodiments, a dose contains from 100-500 mg of the peptide. In some embodiments, a dose contains from 200-300 mg of the peptide. The term, “unit dosage form,” refers to physically discrete units suitable as unitary dosages for human subjects and other mammals, each unit containing a predetermined quantity of active material calculated to produce the desired therapeutic effect, in association with a suitable pharmaceutical diluents or excipients. These can be administered to humans, domestic pets, livestock, or other animals with a pharmaceutically-acceptable diluents or excipients, in unit dosage form. Administration can be topical, intranasal, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration. Formulations for oral use include tablets containing the active ingredient(s) in a mixture with non-toxic pharmaceutically-acceptable excipients. These excipients can be, for example, inert diluents or fillers (e.g., sucrose and sorbitol), lubricating agents, glidams, and antiadhesives (e.g., magnesium stearate, zinc stearate, stearic acid, silicas, hydrogenated vegetable oils, or talc).

The active therapeutic formulation of the invention can be provided in lyophilized form for reconstituting, for instance, in isotonic, aqueous, or saline buffers for parental, subcutaneous, intradermal, intramuscular, or intravenous administration. The subject composition of the invention can also be administered to the patient in need of a therapeutic peptide by liquid preparations for orifice, e.g. oral, intraaural, nasal, or sublingual, administration such as suspensions, syrups or elixirs. The subject composition of the invention can also be prepared for oral administration such as capsules, tablets, and pills, as well as chewable solid formulations. The subject composition of the invention can also be prepared as a cream for dermal administration such as liquid, viscous liquid, paste, or powder. The subject composition of the invention can also be prepared as powder for lung administration with or without aerosolizing component. The composition of the invention can be prepared as a drop, for example, an eardrop.

The presently disclosed compositions can be used for delivery in oral, intraaural, intranasal, sublingual, intraduodenal, subcutaneous, buccal, intracolonic, rectal, vaginal, mucosal, pulmonary, transdermal, intradermal, parenteral, intravenous, intramuscular and ocular forms as well as being able to traverse the blood-brain barrier.

### Formulcitions

The peptide-based compositions of the present invention can be administered by various means, depending on their intended use. For example, if compositions of the present invention are to be administered orally, they can be formulated as tablets, capsules, granules, powders or syrups. Alternatively, formulations of the present invention can be administered parenterally as injections (intravenous, intramuscular or subcutaneous), drop infusion preparations or suppositories. The peptide-based compositions can also be administered into deep lung by aerosolizing the composition into 1-5 um particle either with or without addition of aerosolizing excipient. For application by the ophthalmic mucous membrane route, compositions of the present invention can be formulated as eye drops or eye ointments. Aural pharmaceutical compositions can be formulated as eardrops, ointments, creams, liquids, gels, or salves for application to the ear, either internally or superficially. These formulations can be prepared by conventional means, and, if desired, the compositions can be mixed with any conventional additive, such as an excipient, a binder, a disintegrating agent, a lubricant, a solubilizing agent, a suspension aid, an emulsifying agent or a coating agent.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants can be present in the formulated agents.

Subject peptide-based compositions can be suitable for oral, intra-aural, nasal, topical (including buccal and sublingual), rectal, vaginal, aerosol and/or parenteral administration. The formulations of the peptide-based compositions can conveniently be presented in unit dosage form and can be prepared by any pharmacy method. The amounts of composition that can be combined with other excipients to produce a single dose can vary depending upon the subject being treated, and the particular mode of administration.

Formulations suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention can also be administered as a bolus, electuary, or paste.

In solid dosage forms for oral administration (capsules, tablets, pills, dragees, powders, and granules), the subject peptide composition is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as carboxymethyl cellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as acetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the peptide compositions can also comprise buffering agents. Solid compositions of a similar type can also be employed as fillers in soft and hard-filled gelatin capsules using such excipients as lactose or milk sugars, as well as high molecular weight polyethylene glycols.

A tablet can be made by compression or molding, optionally with one or more accessory ingredients. Compressed tablets can be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets can be made by molding in a suitable machine a mixture of the subject composition moistened with an inert liquid diluent. Tablets, and other solid dosage forms, such as dragees, capsules, pills and granules, can optionally be scored or prepared with coatings and shells, such as enteric coatings.

Liquid dosage forms for oral administration include pharmaceutically-acceptable emulsions, microemulsions, gels, solutions, suspensions, syrups and elixirs. The liquid dosage peptide formulation can contain inert diluents, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspension dosage of the peptide formulation can contain suspending agents as, for example, ethoxylated isostearyl alcohols, polyoxyethylene sorbitol and sorbite esters, microcrystalline cellulose, aluminum metahydroxide, bentonite, agar-agar and tragacanth, and mixtures thereof.

The peptide formulations for rectal or vaginal administration can be presented as a suppository, which can be prepared by mixing a peptide with one or more suitable carriers and other excipients comprising, for example, cocoa butter, polyethylene glycol, a suppository wax or a salicylate, and which is solid at room temperature, but liquid at body temperature and, therefore, will melt in the body cavity and release peptide. Formulations which are suitable for vaginal administration also include pessaries, tampons, creams, gels, pastes, foams, or spray formulations containing excipients.

The peptide dosage formulations for transdermal administration of a subject composition includes drops, powders, sprays, ointments, pastes, creams, lotions, gels, solutions, patches and inhalants. The active component can be mixed under sterile conditions with a pharmaceutically-acceptable carrier, and with any preservatives, buffers, or propellants which can be required.

The ointments, pastes, creams and gels can contain, in addition to a subject composition, excipients, such as animal and vegetable fats, oils, waxes, paraffin, starch, tragacanth, cellulose derivatives, polyethylene glycols, silicones, bentonite, silicic acid, talc and zinc oxide, or mixtures thereof. The peptide compositions of the present invention can also be in the form of baby wipes.

Powders and sprays can contain, in addition to a subject composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The peptide compositions of the present invention can alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers can be used because they minimize exposing the peptide to shear, which can result in degradation of the peptides contained in the subject compositions.

Ordinarily, an aqueous aerosol is made by formulating an aqueous solution or suspension of a subject composition together with conventional pharmaceutically-acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which can be reconstituted into sterile injectable solutions or dispersions just prior to use, which can contain antioxidants, buffers, bacteriostats, solutes which render the formulation isotonic with tre blood of the intended recipient or suspending or thickening agents.

Examples of suitable aqueous and non-aqueous carriers which can be employed in the pharmaceutical compositions of the invention include water, ethanol, polyols (such as glycerol, propylene glycol, and polyethylene glycol), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In some embodiments, the peptide inhibitor(s) or peptide activator(s) are dissolved in a saline solution, such as normal saline (0.9% NaCl). The peptide inhibitor(s) can also be dissolved in organ preservation solutions, such as Krebs-Henseleit solution, UW solution, St. Thomas II solution, Collins solution, Stanford solution, and the like. The solution may also contain one or more of sodium (Na+), potassium (K+), calcium (Ca²⁺), magnesium (Mg²⁺), glutamate, arginine, adenosine, mannitol, glutathione, raffinose, and lactobionic acid in concentrations such as about 4-7 mM, about 0.2-0.3 mM, about 108-132 mM, about 13-16 mM, about 18-22 mM, about 2-4 mM, about 0.5-1 mM, about 27-33 mM, about 0.9-1.1 mM, about 2.7-3.3 mM, about 25-35 mM, and about 80-120 mM, respectively. Na⁺ can be in the form of NaOH; K⁺ can be in the form of KCl and/or KH₂PO₄, in ratios such as about 2-3.5 mM KCl and about 2-3.5 mM KH₂PO₄; Ca²⁺ can be in the form of CaCl₂; and Mg²⁺ can be in the form of MgCl₂. The solution can be maintained at physiological pH of about 7.2-7.4.

### Treatment

The compounds of the present invention can be useful for treatment of reperfusion injury following ischemia. Conditions associated with ischemia can include, for example, acute myocardial infarction (AMI), stroke, including perinatal stroke, hemorrhagic shock, intestinal ischemia, diabetic retinal neuropathy, emergency coronary surgery for failed percutaneous transluminal coronary angioplasty (PCTA), any vascular surgery with blood vessel cross clamping (e.g. of aorta, leading to skeletal muscle ischemia), pancreatitis after manipulation of pancreatic or bile duct, or organ transplantation, including heart, liver, kidney, lung, and pancreas transplantation.

In an embodiment, the solution containing the compound of the present invention can be used during all phases of an organ, especially the heart, transplant, including, but are not limited to, 1) isolating of the organ from the donor (cardioplegic solution); 2) preserving the organ (hypothermic storage/transport); and 3) re-implanting the organ in the recipient (reperfusion solution). The solution can also be used to attenuate or reduce organ damage, e.g. due to ischemia, by contact of the organ with the compound of Formula I. The protective effect may be before, during, or immediately after a surgical procedure on the organ, such as angioplasty, cardiac bypass or any procedure resulting in transient tissue ischemia. The compound is present in the solution at about 1 nM to about 20 μM.

In use, the compound of the present invention may be placed in contact with the organ to protect it from ischemic injury. The organ may be placed in contact with the compound of Formula I by soaking in the solution. Alternatively, the organ may be perfused with the solution containing the compound of Formula I. The contact of the compound of Formula I with the organ may be in vivo, in vitro, or ex vivo.

The compound of the present invention can also be used in a perfusion solution or a preservation solution. As a perfusion solution, it is pumped into the vasculature of the organ to protect the organ tissues and cells. As a preservation solution, it serves as a bathing solution into which the organ is submerged. In some embodiments, the organ is perfused with and submerged in the present solution. Further, the present solution also serves as a reperfusion solution upon restoration of blood flow to the organ after ischemia. During perfusion or reperfusion, especially for the heart, it is preferred that the organ be perfused with the solution containing the compound at a rate of about 1 mL/min/g of organ weight for about 5 min. The perfusion rate can be varied, but it should not exceed about 25 mL/min/g of organ weight. Overall, the perfusion rate should not be so high as to impose undue pressure on the vasculature of the organ.

### Dosoges

The dosage of any peptide of the present invention will vary depending on the symptoms, age and body weight of the patient, the nature, location, and severity of the reperfusion injury to be treated or prevented, the route of administration, and the form of the composition. Any of the subject formulations can be administered in a single dose or in divided doses. Also, the present invention contemplates mixtures of more than one subject peptide, as well as other therapeutic agents.

In certain embodiments, the dosage of the subject peptide can be in the range of about 0.01 ng to about 10 g per kg body weight, specifically in the range of about 1 ng to about 0.1 g per kg, and more specifically in the range of about 100 ng to about 20 mg per kg.

An effective dose or amount, and any possible effects on the timing of administration of the formulation, can need to be identified for any particular peptide of the present invention. This end can be accomplished by routine experiment as described herein, using one or more groups of animals, or in human trials if appropriate. The effectiveness of any peptide and method of treatment or prevention can be assessed by administering the supplement and assessing the effect of the administration by measuring one or more indices associated with the neoplasm of interest, and comparing the post-treatment values of these indices to the values of the same indices prior to treatment.

The precise time of administration and amount of any particular peptide that will yield the most effective treatment in a given patient can depend upon the activity, pharmacokinetics, and bioavailability of a particular peptide, physiological condition of the patient (including age, sex, disease type and stage, general physical condition, responsiveness to a given dosage and type of medication), and route of administration. The guidelines presented herein can be used to optimize the treatment, e.g., determining the optimum time and/or amount ofadministration, which will require no more than routine experimentation consisting of monitoring the subject and adjusting the dosage and/or timing.

While the subject is being treated, the health of the subject can be monitored by measuring one or more of the relevant indices at predetermined times during a 24-hour period. Treatment, including supplement, amounts, times of administration and formulation, can be optimized according to the results of such monitoring. The patient can be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy can continue for several months or even years, with a minimum of one month being a typical length of therapy for humans. Adjustments to the amount(s) of peptide administered and possibly to the time of administration can be made based on these reevaluations.

Treatment can be initiated with smaller dosages which are less than the optimum dose of the peptide. Thereafter, the dosage can be increased by small increments until the optimum therapeutic effect is attained.

The combined use of several peptides of the present invention, or alternatively other peptides, can reduce the required dosage for any individual component because the onset and duration of effect of the different components can be complimentary. In such combined therapy, the different peptides carr be delivered together or separately, and simultaneously or at different times within the day.

Toxicity and therapeutic efficacy of subject peptides can be determined by standard pharmaceutical procedures in cell cultures or experimental animals, e.g., for determining the LD₅₀ and the ED₅₀. Although peptides that exhibit toxic side effects can be used, care should be taken to design a delivery system that targets the peptides to the desired site in order to reduce side effects.

The data obtained from cell culture assays and animal studies can be used in formulating a range of dosage for use in humans. The dosage of any supplement, or alternatively of any components therein, lies within a range of circulating concentrations that include the ED₅₀, with little or no toxicity. The dosage can vary within this range depending upon the dosage form employed and the route of administration utilized. For peptides of the present invention, the therapeutically effective dose can be estimated initially from cell culture assays. A dose can be formulated in animal models to achieve a circulating plasma concentration range that includes the IC₅₀ (i.e., the concentration of the test peptide which achieves a half-maximal inhibition of symptoms) as determined in cell culture. Such information can be used to more accurately determine useful doses in humans. Levels in plasma can be measured, for example, by high performance liquid chromatography (HPLC).

The compound of the present invention can be present in a composition in a range of from about 1 mg to about 2000 mg; from about 5 mg to about 1000 mg, from about 10 mg to about 25 mg to 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound of if pre sent invention can be present a compositionn an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

Without further description, it is believed that orae of ordinary skill in the art can, using the preceding description and the following illustrative example, make and utilize the compounds of the present invention and practice the claimed methods. The following example is given to illustrate the present invention. It should be understood that the invention is not to be limited to the specific conditions or details described in the example.

### EXAMPLE 1

### Mitigation of Reperfusion Injury in Isolated Rat Hearts

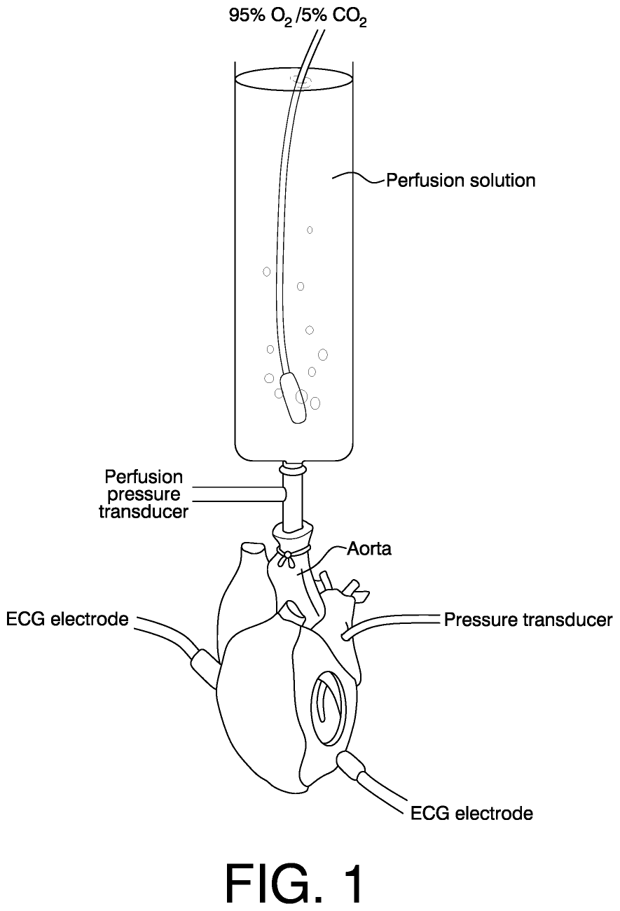

A series of protein kinase inhibitors were evaluated in the attenuation of myocardial ischemia/reperfusion (I/R) injury in isolated rat hearts. Rat hearts were isolated from anesthetized male Sprague Dawley rats (275-325 g), placed in ice-cold Krebs' buffer, and placed onto a Langendorff heart apparatus and retro perfused with Krebs' buffer at a constant pressure of 80 mmHg (FIG. 1). A pressure transducer measures heart rate, left ventricular end-systolic pressure, end-diastolic pressure, dP/dt max, and dP/dt min. Coronary flow is measured via a flow probe connected to the perfusion tubing. Cardiac function was recorded throughout the experimental protocol using a pressure transducer inserted into the left ventricle (15 min baseline; global I(30 min)/R(50 min)). Selected peptides of TABLE 1 or vehicle (control [0.05 to 0.2% Dimethylsulfoxide) were given during the first 5 min of reperfusion. All peptide inhibitors or activators were dissolved in 28% DMSO and the final concentration of DMSO with peptides or without peptides (control I/R hearts) delivered to all hearts was 0.06%. Statistical analysis was performed using ANOVA and Bonferroni-Dunn post-hoc test.

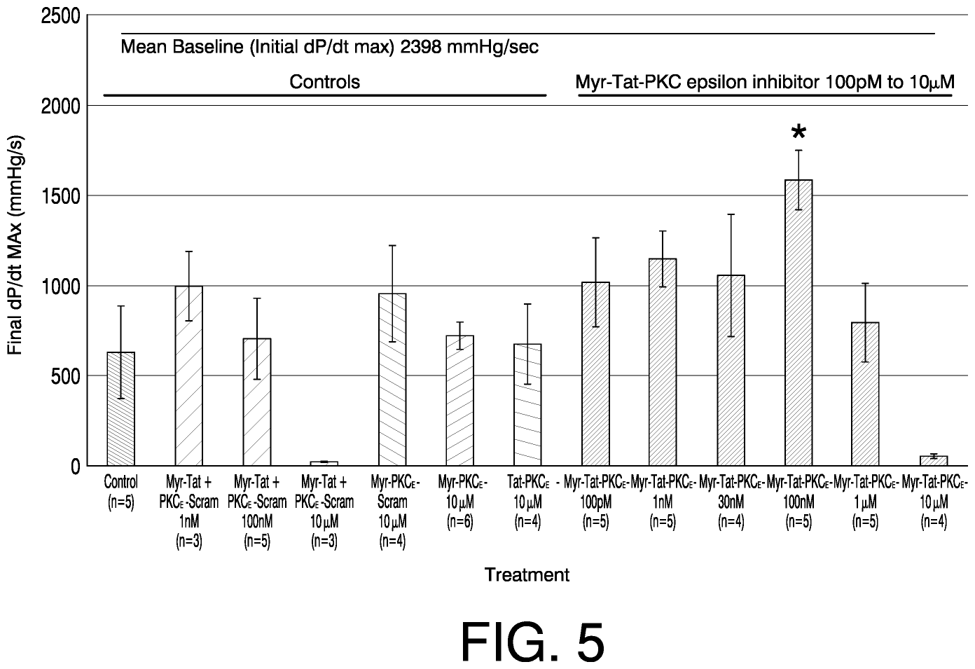

At the end of the experimental protocol, hearts were removed from the Langendorff apparatus, and frozen for 30 min at −20° C. Hearts were sectioned (2 mm) from base to apex and placed in 1% TTC for ˜10 min at 37° C. Viable tissue stained red and infarcted tissue was unstained (pale). Total tissue sections were weighed and infarcted tissue was separated from viable tissue and weighed to determine infarct size (i.e. infarct tissue weight/total tissue weight). Initial baseline (before ischemia) and final (at 50 minutes post-reperfusion) average dP/dtₘₐₓ in ex vivo rat hearts as categorized by exposure to various concentrations of the compounds are summarized in FIG. 5.

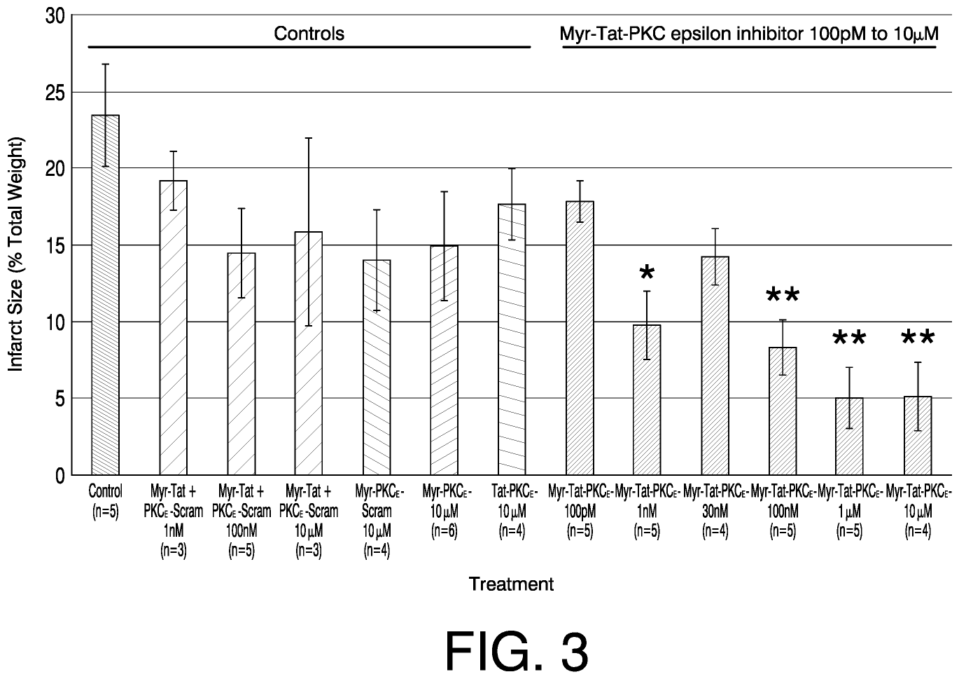

The resulting myocardial infarct size (non-viable/total tissue) in the isolated rat hearts (ex vivo) are summarized in FIG. 3 (*P<0.05; **P<0.01 vs. control I/R hearts treated with vehicle agent dimethyl suboxide (DMSO) at a final concentration of 0.06%).

Initial baseline (before ischemi and final values (at 50 minutes post-reperfusion for left ventricular end-systolic pressure (LVESP), left ventricular end-diastolic pressure (LVEDP), left ventricular developed pressure (LVDP; LVDP=LVESP−LVEDP), dP/dtₘᵢₙ, dP/dtₘₐₓ, heart rate, and coronary flow were measured and administration of various concentrations of selected inhibitors and scrambled controls are provided in TABLE 2.

TABLE 2

| | | Myr + | Myr + | Myr + | Myr + | Myr + | Myr + |
|---|---|---|---|---|---|---|---|
| | | TAT + | TAT + | TAT + | TAT + | TAT + | TAT + |
| | Control | PKCε- | PKCε- | PKCε- | PKCε- | PKCε- | PKCε- |
| | (DMSO) | 100 pM | 1 nM | 30 nM | 100 nM | 1 μM | 10 μM |
| | (n = 5) | (n = 5) | (n = 5) | (n = 4) | (n = 5) | (n = 5) | (n = 4) |
| Initial LVESP | 100 ± 3 | 100 ± 3 | 101 ± 5 | 99 ± 2 | 98 ± 3 | 104 ± 6 | 101 ± 4 |
| (mmHg) | | | | | | | |
| Initial LVEDP | 6 ± 1 | 10 ± 0.4 | 10 ± 1 | 11 ± 1 | 9 ± 1 | 10 ± 2 | 8 ± 2 |
| (mmHg) | | | | | | | |
| Initial LVDP | 93 ± 3 | 89 ± 3 | 91 ± 4 | 88 ± 2 | 89 ± 4 | 94 ± 5 | 92 ± 3 |
| (mmHg) | | | | | | | |
| Final LVESP | 105 ± 12 | 94 ± 14 | 101 ± 9 | 108 ± 3 | 114 ± 11 | 101 ± 6 | 69 ± 17 |
| (mmHg) | | | | | | | |
| Final LVEDP | 73 ± 4 | 47 ± 10 | 52 ± 9 | 50 ± 15 | 48 ± 13 | 60 ± 12 | 68 ± 17 |
| (mmHg) | | | | | | | |
| Final LVDP | 33 ± 12 | 47 ± 11 | 49 ± 8 | 48 ± 10 | 66 ± 6 | 40 ± 15 | 1 ± 0.2 |
| (mmHg) | | | | | | | |
| Initial dP/dtmax | 2478 ± 87 | 2457 ± 37 | 2440 ± 72 | 2344 ± 22 | 2528 ± 133 | 2493 ± 128 | 2540 ± 158 |
| (mmHg/s) | | | | | | | |
| Final dP/dtmax | 632 ± 254 | 1021 ± 246 | 1147 ± 156 | 1059 ± 336 | 1585* ±164 | 794 ± 220 | 54 ± 14 |
| (mmHg/s) | | | | | | | |
| Initial dP/dtmin | −1693 ± 15 | −1726 ± 49 | −1674 ± 106 | −1588 ± 18 | −1807 ± 125 | −1786 ± 97 | −1772 ± 141 |
| (mmHg/s) | | | | | | | |
| Final dP/dtmin | −464 ± 163 | −688 ± 142 | −733 ± 68 | −733 ± 99 | −981 ± 135 | −616 ± 122 | −56 ± 14 |
| (mmHg/s) | | | | | | | |
| Initial Coronary | 18 ± 2 | 18 ± 2 | 18 ± 1 | 16 ± 1 | 20 ± 2 | 21 ± 1 | 18 ± 2 |
| Flow (mL/min) | | | | | | | |
| Final Coronary | 9 ± 2 | 10 ± 1 | 10 ± 1 | 9 ± 1 | 11 ± 2 | 10 ± 3 | 0 |
| Flow (mL/min) | | | | | | | |
| Initial Heart | 289 ± 10 | 285 ± 6 | 293 ± 13 | 271 ± 7 | 301 ± 10 | 274 ± 15 | 285 ± 24 |
| Rate (BPM) | | | | | | | |
| Final Heart | 253 ± 11 | 252 ± 8 | 266 ± 6 | 261 ± 11 | 254 ± 12 | 296 ± 86 | 811 ± 79 |
| Rate (BPM) | | | | | | | |
| Infarct Size (%) | 23 ± 3 | 18 ± 1 | 10 ± 2* | 14 ± 2 | 8 ± 2** | 5 ± 2** | 5 ± 2** |
| | | Myr + | Myr + | Myr + | | | |
| | | TAT + | TAT + | TAT + | | | Myr + |
| | | PKCε- | PKCε- | PKCε- | TAT + | Myr + | PKCε- |
| | | Scram. | Scram. | Scram. | PKCε- | PKCε- | Scram. |
| | | 1 nM | 100 nM | 10 μM | 10 μM | 10 μM | 10 μM |
| | | (n = 3) | (n = 5) | (n = 3) | (n = 4) | (n = 5) | (n = 4) |
| | Initial LVESP | 92 ± 1 | 100 ± 4 | 92 ± 0.3 | 93 ± 3 | 95 ± 5 | 96 ± 6 |
| | (mmHg) | | | | | | |
| | Initial LVEDP | 6 ± 0.4 | 9 ± 2 | 6 ± 1 | 7 ± 2 | 6 ± 1 | 9 ± 2 |
| | (mmHg) | | | | | | |
| | Initial LVDP | 87 ± 1 | 91 ± 5 | 86 ± 1 | 86 ± 5 | 90 ± 5 | 87 ± 4 |
| | (mmHg) | | | | | | |
| | Final LVESP | 103 ± 14 | 93 ± 9 | 72 ± 8 | 100 ± 13 | 104 ± 6 | 107 ± 4 |
| | (mmHg) | | | | | | |
| | Final LVEDP | 53 ± 4 | 57 ± 10 | 70 ± 10 | 63 ± 2 | 58 ± 3 | 56 ± 8 |
| | (mmHg) | | | | | | |
| | Final LVDP | 50 ± 11 | 36 ± 12 | 3 ± 2 | 37 ± 12 | 46 ± 8 | 51 ± 9 |
| | (mmHg) | | | | | | |
| | Initial dP/dtmax | 2267 ± 87 | 2300 ± 189 | 2309 ± 69 | 2408 ± 126 | 2294 ± 108 | 2307 ± 55 |
| | (mmHg/s) | | | | | | |
| | Final dP/dtmax | 993 ± 195 | 706 ± 223 | 27 ± 9 | 678 ± 223 | 723 ± 84 | 958 ± 265 |
| | (mmHg/s) | | | | | | |
| | Initial dP/dtmin | −1490 ± 82 | −1545 ± 117 | −1604 ± 71 | −1735 ± 149 | −1548 ± 92 | −1571 ± 72 |
| | (mmHg/s) | | | | | | |
| | Final dP/dtmin | −760 ± 210 | −677 ± 222 | −24 ± 8 | −723 ± 84 | −694 ± 167 | −727 ± 133 |
| | (mmHg/s) | | | | | | |
| | Initial Coronary | 15 ± 1 | 21 ± 6 | 18 ± 1 | 20 ± 2 | 17 ± 2 | 19 ± 3 |
| | Flow (mL/min) | | | | | | |
| | Final Coronary | 7 ± 2 | 12 ± 4 | 3 ± 0.1 | 12 ± 4 | 10 ± 1 | 8 ± 2 |
| | Flow (mL/min) | | | | | | |
| | Initial Heart | 278 ± 10 | 271 ± 16 | 275 ± 12 | 285 ± 10 | 258 ± 8 | 278 ± 10 |
| | Rate (BPM) | | | | | | |
| | Final Heart | 225 ± 1 | 295 ± 29 | 684 ± 167 | 242 ± 18 | 233 ± 10 | 236 ± 11 |
| | Rate (BPM) | | | | | | |
| | Infarct Size (%) | 19 ± 2 | 14 ± 3 | 16 ± 6 | 18 ± 2 | 15 ± 4 | 14 ± 3 |

*p < 0.05 vs. Control (final or infarct size)
**p < 0.01 vs. Control (final or infarct size) ANOVA post-hoc analysis using Bonferroni-Dunn.

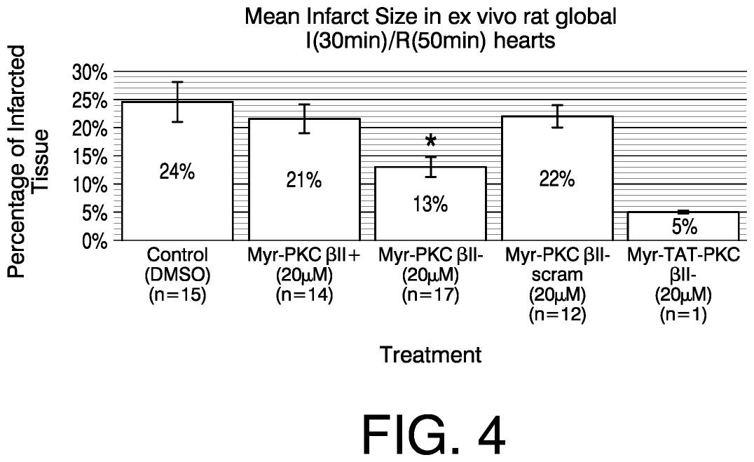

10 μM Myr-PKCε inhibitor (Myr+PKCε−) and 10 μM TAT-PKCε inhibitor (TAT+PKCε−) did not reduce infarct size compared to control I/R hearts (FIG. 3). This result suggests that Myr−TAT conjugation is superior to Myr or TAT conjugation in the reduction of cell death (i.e. infarct size). The tissue-salvaging effects of Myr+TAT+PKCε− are effective down to 1 nM concentration, which represents a 10,000× decrease in minimum effective concentration as compared to Myr+PKCε−. This was an unexpected finding and not obvious that potency would be increased by 10,000× compared to Myr or TAT conjugated PKCE inhibitor. The Myr+TAT+PKCε inhibitor also has demonstrated dramatically enhanced solubility over a wide range of concentrations (up to 100 mM) over Myr−PKCε inhibitor which is limiting beyond 10 mM). As shown in FIG. 4, Myr+Tat+PKCβII− also showed a reduction of infarct size in ex vivo rat I/R hearts (5%, n=1) compared to Myr−PKCβII− (13±2%, n=17) or Myr−PKCβII− scrambled (22±2%, n=12) or control (24±3%, n=15) hearts.

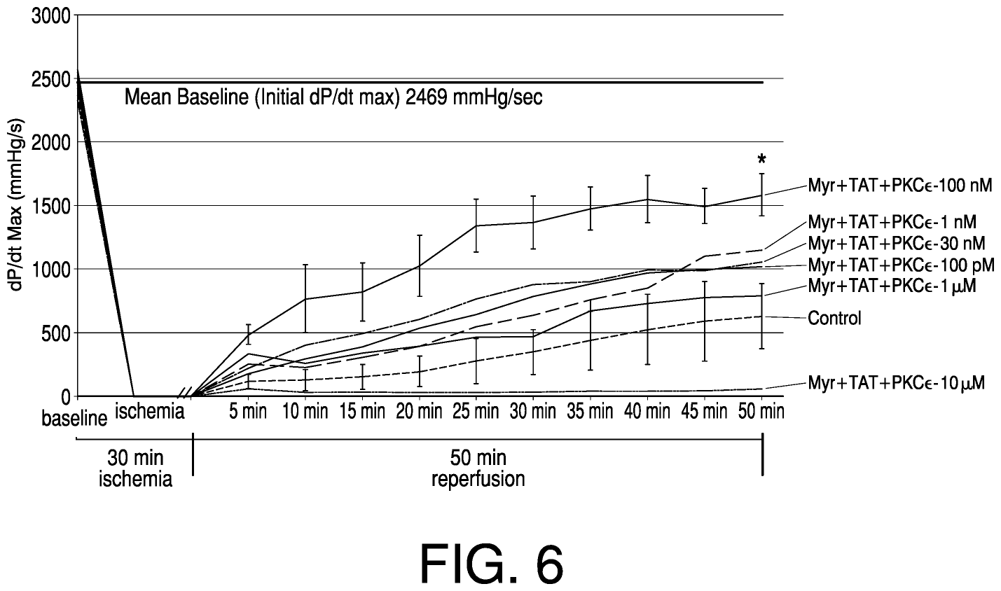

100 nM Myr+TAT+PKCε− given at the beginning of reperfusion for 5 minutes significantly restored post-reperfused cardiac function (25 to 50 min) compared to control I/R hearts (see TABLE 2 and FIGS. 5 and 6). In contrast, Myr+PKCε− exhibited a similar effect only at 100× greater concentration. Moreover, 10 μM Myr+PKCε− was ineffective to restore post-reperfused cardiac function, and therefore lower concentrations would not be expected to restore post-reperfused cardiac function. 10 μM and 1 μM Myr+TATPKCε− had the most robust effect on reducing infarct size. This result suggests that Myr−TAT conjugation has a synergistic effect to salvage cardiac tissue and enhance the potency (10,000×) of the cargo sequence (i.e., the PKCε inhibitor) to reduce infarct size and restore post-reperfused cardiac function (100×). 10 μM Myr+TAT+PKCε− and 10 μM Myr+TAT+PKCε− scrambled had the most cardio depressive effect on post-reperfused cardiac function compared to all other groups (TABLE 2). This result suggests that 10 μM concentrations of Myr+TAT+PKCε− and Myr+TAT+PKCε− scrambled peptides might cause stunning of the heart during reperfusion. However, the observation that 10 μM Myr+TAT+PKCε− reduced infarct size suggests that the proper cargo sequence is effective to preserve tissue viability. While the 10 μM concentration of Myr+TAT+PKCε− caused stunning, this is 10,000 times the concentration needed to produce a 50% reduction in infarct size.

Collectively, the results suggest that Myr−TAT conjugation on peptides that have intracellular targets is superior to Myr alone or TAT LONW conjugation. Further, the results provide the basis for a platform technology to augment the efficacy of peptides or small molecules that have intracellular targets.

### EXAMPLE 2

### Suppression of Superoxide (SO) Release in Rat Polymorphonuclear Leukocytes

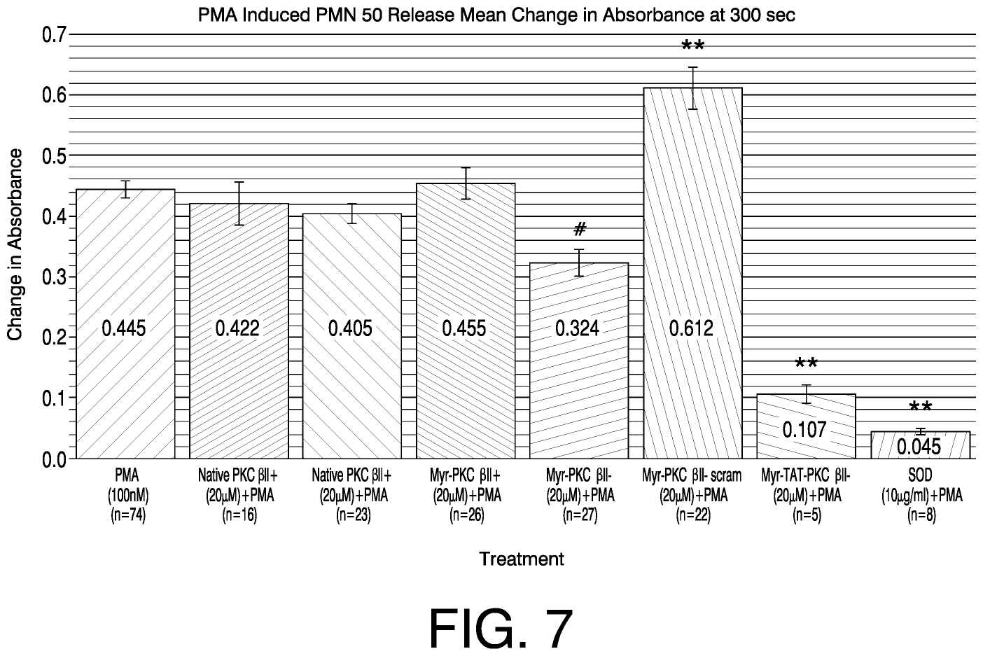

Change in absorbance at 550 nm via ferricytochrome c reduction by superoxide after stimulation with 100 nM phorbol 12-myristate 13-acetate (PMA) from time 0 to 390 sec was monitored in rat polymorphonuclear leukocytes (PMNs) (5×10⁶) incubated for 15 min at 37° C. in the presence/absence of unconjugated PKCβII activator (PKCβII+) (SVEIWD (SEQ ID NO: 23)) or inhibitor (PKCβII−) (SLNPEWNET(SEQ ID NO: 5)), Myr−PKCβII+/−, Myr−PKCβII− scram (Myr-WNPESLNTE (Myristoylated SEQ ID NO: 17), Myr−TAT−PKC|II− (Myristoylated SEQ ID NO: 15) (all 20 μM) or superoxide dismutase (SOD; 10 μg/mL) (FIG. 7). Stock concentrations of compounds were dissolved in DMSO to yield a final concentration not greater that 0.2%. #: p<0.05 vs PMA, Native PKC βII+, Myr−PKC βII+ and Myr−TAT−PKC βII−; **: p<0.01 vs. all groups except Myr−TAT−PKC βII− or SOD; ++: p<0.01 vs. all groups. ANOVA post-hoc analysis was conducted using Bonferroni-Dunn.

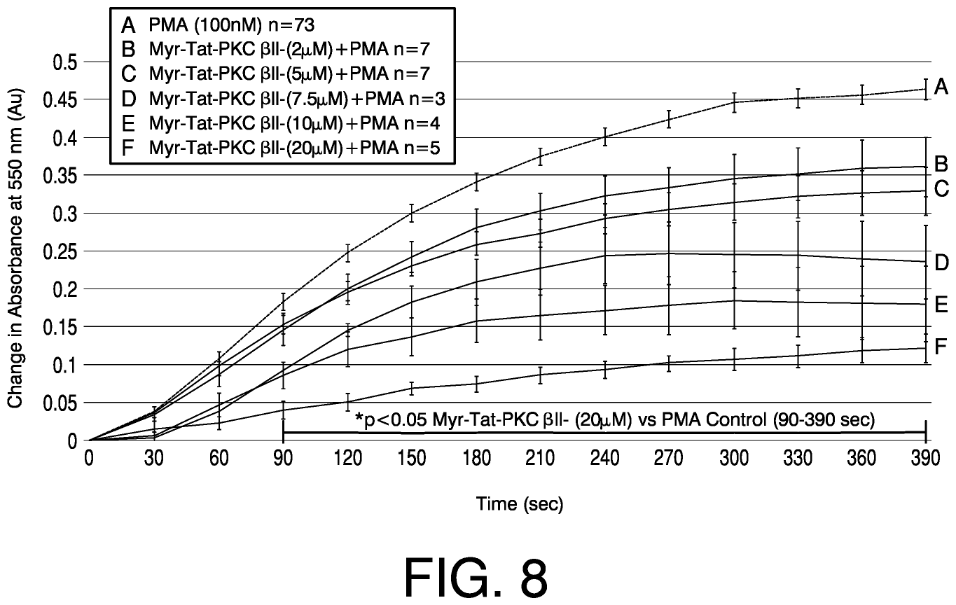

The time course of changes in absorbance are summarized in FIGS. 8 (concentration response of Myr+TAT PKCβII−) and 9 (comparison of Myr+TAT+PKCβII− and Myr−PKCβII−, Myr−PKCβII− scrambled, and PKCβII− analogues and PMA controls). The peak response was reached by 300 seconds and maintained a plateau to 390 seconds, Myr+TAT+PKCβII− (20 μM; n=5) reduced phorbol 12-myristate 13-acetate (PMA, 100 nM) induced leukocyte superoxide release by 80%—a degree that was greater than all other study groups (p<0.01). In contrast, the Myr+PKCβII− (20 μM; n=27) reduced leukocyte superoxide release by only 30% (20 μM; n=27), again supporting the concept that the dual Myr−TAT conjugation produced greater results and reduced leukocyte superoxide release by ˜80% and ˜65% as compared to untreated PMA controls (n=74) and Myr+PKCβII− (20 μM; n=27), respectively.

Myr−PKCβII-scram increased. PMA induced leukocyte superoxide release compared to all groups that also included native unconjugated PKCβII+ and PKCβII− peptides (p<0.01). Unconjugated (Native) PKCβII− did not exert a significant inhibition of leukocyte superoxide release compared to controls (i.e. PMA+0.2% DMSO only). Superoxide dismutase (SOD; 10 ug/ml; n=8), used herein as a positive control, attenuated leukocyte superoxide release by ˜94% compared to all groups. The effects of Myr+TAT+PKCβII− were virtually identical to the SOD standard. The inhibition of leukocyte superoxide release by Myr−TAT−PKCβII− suggests that this Myr−TAT conjugation to piccpm elicits tissue salvaging effects in myocardial I/R injury. Studies with Myr−PKCβII− indicate a correlation with reduction in leukocyte superoxide and hindlimb I/R induced hydrogen peroxide release and cardioprotection m myocardial I/R injury.

### EXAMPLE 3

### Expected Effect of Myr−Tat Conjugated PKC Modulators in Mitigating Ischemia/Reperfusion Injury in Male Yorkshire Pigs

The protective effect on heart function and injury (infarct size) of selected compounds of TABLE 1 are expected in an I/R injury in vivo model in male Yorkshire cross pigs when evaluated. Male pigs are used to eliminate the possible influence of estrogen in the study. Study cohorts are summarized in TABLE 3.

TABLE 3

| Group | Dose (mg/kg) | Compound |
|---|---|---|
| A | 0.2 | Myr + TAT + PKCε- scrambled |
| B | 0.2 | Myr + TAT + PKCε- |
| C | 4.5 | Myr + TAT + PKCβII- scrambled |
| D | 4.5 | Myr + TAT + PKCβII- |

Castrated male Yorkshire pigs (˜45 kg, n=14) are subjected to 1 hour of regional left anterior descending artery (LAD) occlusion located at the second branch of the LAD coronary artery by catheter balloon inflation/clamping, and then are reperfused for 3 hours (deflation of balloon/removal of clamps). The test and control compounds of TABLE 3 are administered as an intravenous bolus at the time of reperfusion via the jugular vein. Echocardiography and troponin I and creatine phosphokinase measurements are used to assess cardiac function and heart injury throughout the experiment. Infarct size is determined using Evans dye (area at risk) and 1% triphenyltetrazolium chloride (TTC) to determine viable (red stain) vs. infarcted tissue (unstained) using computerized planimetry in excised hearts that are sectioned (8 mm) from base to apex at the end of the experimental protocol (i.e., 3 hr. reperfusion).

Selected Myr−TAT conjugated peptides of TABLE 1 are expected to produce a reduction in plasma troponin I and CPK (indices of heart injury) and infarct size accompanied by an increase in post-reperfused cardiac function (i.e., ejection fraction) throughout the 3 hour reperfusion period compared to control scrambled Myr−TAT conjugated peptides. The Myr−TAT conjugated peptides are additionally expected to decrease expression of PKCε or PKCβII plasma membrane localization compared to control scrambled peptide using frozen heart sections from the porcine I/R experiments. Immunohistochemical localization of PKCε and PKCβII is also undertaken.

TABLE 4
SEQUENCE LISTING

| | Sequence | SEQ |
|---|---|---|
| Sequence | (amino to | ID |
| description | carbonyl) | NO |
| TAT Subdomain 1 | YGRKKRRQRRR | 1 |
| TAT Subdomain 2 | GRKKRRQRRR | 2 |
| TAT Subdomain 3 | GRKKRRQRRR | 3 |
| PKCϵ- | EAVSLKPT | 4 |
| PKCβII- | SLNPEWNET | 5 |
| PKCδ+ | MRAAEDPM | 6 |
| PKCζ- version 1 | SIYRRGARRWRKL | 7 |
| PKCζ- version 2 | SIYRRGARRWRKLYR | 8 |
| | AN | |
| Tetramer linker 1 | CCCC | 9 |
| Tetramer linker 2 | GGGG | 10 |
| Tetramer linker 3 | CGGC | 11 |
| Tetramer linker 4 | GCCG | 12 |
| Tetramer linker 5 | CGCG | 13 |
| Tetramer linker 6 | GCGC | 14 |
| TAT + CC + PKCβII- | YGRKKRRQRRRCCSL | 15 |
| | NPEWNET | |
| TAT + CC + PKCβII- | YGRKKRRQRRRCCW | 16 |
| scrambled | NPESLNTE | |
| PKCβII- scrambled | WNPESLNTE | 17 |
| TAT + CC + PKCϵ- | YGRKKRRQRRRCCEA | 18 |
| | VSLKPT | |
| TAT + CC + PKCϵ- | YGRKKRRQRRRCCLS | 19 |
| scrambled | ETKPAV | |
| PKCϵ- scrambled | LSETKPAV | 20 |
| TAT + CC + PKCδ+ | YGRKKRRQRRRCCM | 21 |
| | RAAEDPM | |
| TAT + CC + PKCδ+ | YGRKKRRQRRRCCA | 22 |
| scrambled | MEADPMR | |
| PKCβII+ | SVEIWD | 23 |
| TAT + CC + PKCζ- | YGRKKRRQRRRCCSI | 24 |
| version 1 | YRRGARRWRKL | |
| TAT + CC + PKCζ- | YGRKKRRQRRRCCSI | 25 |
| version 2 | YRRGARRWRKLYRA | |
| | N | |
| PKCδ+ scrambled | AMEADPMR | 26 |
| TAT + GG + PKCϵ- | YGRKKRRQRRRGGEA | 27 |
| | VSLKPT | |
| TAT + GG + PKCβII- | YGRKKRRQRRRGGSL | 28 |
| | NPEWNET | |
| TAT + GG + PKCδ- | YGRKKRRQRRRGGM | 29 |
| | RAAEDPM | |
| TAT + GG + PKCζ- | YGRKKRRQRRRGGSI | 30 |
| version 1 | YRRGARRWRKL | |
| TAT + GG + PKCζ- | YGRKKRRQRRRGG | 31 |
| version 2 | SIYRRGARRWRKLYR | |
| | AN | |
| TAT + CC + PKCζ- | YGRKKRRQRRRCCRL | 32 |
| version 1 scrambled | YRKRIWRSAGR | |
| TAT + CC + PKCζ- | YGRKKRRQRRRCCRL | 33 |
| version 2 scrambled | RYRNKRIWRSAYAGR | |
| TAT + GG + PKCζ- | YGRKKRRQRRRGGRL | 34 |
| version 1 scrambled | YRKRIWRSAGR | |
| TAT + GG + PKCζ- | YGRKKRRQRRRGGRL | 35 |
| version 2 scrambled | RYRNKRIWRSAYAGR | |

Although certain presently preferred embodiments of the invention have been specifically, described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A compound comprising a peptide haying the structure of Formula I
Myr−TAT−L−P (Formula I)
wherein Myr is myristoyl; TAT is the transduction domain of Trans-Activator of Transcription or lipidated adducts thereof; L is a linker peptide; and P is a peptide modulator of PKCε.

2. The compound of claim 1, wherein L has two to four amino acids.

3. The compound of claim 2, wherein L has an amino acid sequence identical to or with at least about 80% identity to any one of SEQ ID NOS: 9-14.

4. The compound of claim 1, wherein TAT has an amino acid sequence identical to or with at least about 80% identity to any one of SEQ ID NOS: 1-3.

5. The compound of claim 1, wherein P is a PKCε inhibitor, PKCβII inhibitor, PKCζ inhibitor, or PKCδ activator.

6. The compound of claim 5, wherein the PKCε inhibitor has an amino acid sequence with at least 80% identity to or identical to SEQ ID NO: 4.

7. The compound of claim 5, wherein the PKCβII inhibitor has an amino acid sequence with at least 80% identity to or identical to SEQ ID NO: 5.

8. The compound of claim 5, wherein the PKCδ activator has an amino acid sequence with at least 80% identity to or identical to SEQ ID NO: 6.

9. The compound of claim 5, wherein the PKCδ inhibitor has an amino acid sequence with at least 80% identity to or identical to any one of SEQ ID NOS: 7-8.

10. The compound of claim 1, wherein the TAT, the linker, and the peptide modulator of PKCε form a peptide haying an amino acid sequence with at least about 80% identity to or identical to any one of SEQ ID NOS: 15, 18, 21, 24, 25, and 27-31.

11. The compound of claim 1, wherein TAT has an amino acid sequence with at least about 80% identity to any one of SEQ ID NOS: 1-3, L has an amino acid sequence with at least about 80% identity to any one of SEQ ID NOS: 9-14, and P has an amino acid sequence with at least 80% identity to any one of SEQ ID NOS: 4-8.

12. The compound of claim 1, wherein TAT has an amino acid sequence identical to any one of SEQ ID NOS: 1-3, L has an amino acid sequence identical to any one of SEQ ID NOS: 9-14, and P has an amino acid sequence identical to any one of SEQ ID NOS: 4-8.

13. A solution for perfusion, preservation, and/or reperfusion for organ preservation comprising the compound of claim 1.

14. The solution of claim 13, wherein the compound of claim 1 is dissolved in saline solution, Krebs-Henseleit solution, University of Wisconsin solution, St. Thomas II solution, Collins solution, or Stanford solution.

15. The solution of claim 13, further comprising sodium (Na⁺), potassium (K⁺), calcium (Ca²⁺), magnesium (Mg²⁺), glutamate, arginine, adenosine, mannitol, allopurinol, glutathione, raffinose, lactobionic acid or combinations thereof.

16. The solution of claim 13, wherein the concentration of the compound is about 1 nM to about 20 μM.

17. A method for preserving an organ for transplantation, protecting an organ from ischemic damage, attenuating organ dysfunction after ischemia, or protecting an organ from damage after isolation from the circulatory system, the method comprising the step of contacting the organ with the compound of claim 1.

18. The method of claim 17, where in the organ in a heart.

19. The method of claim 17, wherein the contacting step involves perfusing the organ with a solution having the compound dissolved therein.

20. The method of claim 19, wherein the perfusing step takes place at a rate of about 1 mL/minute/g organ weight.


SEQUENCE LISTING (35 sequences)

SEQ ID NO: 1 - protein, 11 aa, Human immunodeficiency virus
YGRKKRRQRRR

SEQ ID NO: 2 - protein, 10 aa, Human immunodeficiency virus
GRKKRRQRRR

SEQ ID NO: 3 - protein, 10 aa, Human immunodeficiency virus
GRKKRRQRRR

SEQ ID NO: 4 - protein, 8 aa, Artificial Sequence
  Artificially synthesized
EAVSLKPT

SEQ ID NO: 5 - protein, 9 aa, Artificial Sequence
  Atificially synthesized
SLNPEWNET

SEQ ID NO: 6 - protein, 8 aa, Artificial Sequence
  Artificially synthesized
MRAAEDPM

SEQ ID NO: 7 - protein, 13 aa, Artificial Sequence
  Artificially synthesized
SIYRRGARRWRKL

SEQ ID NO: 8 - protein, 17 aa, Artificial Sequence
  Artificially synthesized
SIYRRGARRWRKLYRAN

SEQ ID NO: 9 - protein, 4 aa, Artificial Sequence
  Artificially synthesized
CCCC

SEQ ID NO: 10 - protein, 4 aa, Artificial Sequence
  Artificially synthesized
GGGG

SEQ ID NO: 11 - protein, 4 aa, Artificial Sequence
  Artificially synthesized
CGGC

SEQ ID NO: 12 - protein, 4 aa, Artificial Sequence
  Artificially synthesized
GCCG

SEQ ID NO: 13 - protein, 4 aa, Artificial Sequence
  Artificially synthesized
CGCG

SEQ ID NO: 14 - protein, 4 aa, Artificial Sequence
  Artificially synthesized
GCGC

SEQ ID NO: 15 - protein, 22 aa, Artificial Sequence
  Human immunodeficiency virus, artificially synthesized
YGRKKRRQRRRCCSLNPEWNET

SEQ ID NO: 16 - protein, 22 aa, Artificial Sequence
  Human immunodeficiency virus, artificially synthesized
YGRKKRRQRRRCCWNPESLNTE

SEQ ID NO: 17 - protein, 9 aa, Artificial Sequence
  Artificially synthesized
WNPESLNTE

SEQ ID NO: 18 - protein, 21 aa, Artificial Sequence
  Human immunodeficiency virus, artificially synthesized
YGRKKRRQRRRCCEAVSLKPT

SEQ ID NO: 19 - protein, 21 aa, Artificial Sequence
  Human immunodeficiency virus, artificially synthesized
YGRKKRRQRRRCCLSETKPAV

SEQ ID NO: 20 - protein, 8 aa, Artificial Sequence
  Human immunodeficiency virus, artificially synthesized
LSETKPAV

SEQ ID NO: 21 - protein, 21 aa, Artificial Sequence
  Human immunodeficiency virus, artificially synthesized
YGRKKRRQRRRCCMRAAEDPM

SEQ ID NO: 22 - protein, 21 aa, Artificial Sequence
  Human immunodeficiency virus, artificially synthesized
YGRKKRRQRRRCCAMEADPMR

SEQ ID NO: 23 - protein, 6 aa, Artificial Sequence
  Artificially synthesized
SVEIWD

SEQ ID NO: 24 - protein, 26 aa, Artificial Sequence
  Human immunodeficiency virus, artificially synthesized
YGRKKRRQRRRCCSIYRRGARRWRKL

SEQ ID NO: 25 - protein, 30 aa, Artificial Sequence
  Human immunodeficiency virus, artificially synthesized
YGRKKRRQRRRCCSIYRRGARRWRKLYRAN

SEQ ID NO: 26 - protein, 8 aa, Artificial Sequence
  Artificially synthesized
AMEADPMR

SEQ ID NO: 27 - protein, 21 aa, Artificial Sequence
  Human immunodeficiency virus, artificially synthesized
YGRKKRRQRRRGGEAVSLKPT

SEQ ID NO: 28 - protein, 22 aa, Artificial Sequence
  Human immunodeficiency virus, artificially synthesized
YGRKKRRQRRRGGSLNPEWNET

SEQ ID NO: 29 - protein, 21 aa, Artificial Sequence
  Human immunodeficiency virus, artificially synthesized
YGRKKRRQRRRGGMRAAEDPM

SEQ ID NO: 30 - protein, 26 aa, Artificial Sequence
  Human immunodeficiency virus, artificially synthesized
YGRKKRRQRRRGGSIYRRGARRWRKL

SEQ ID NO: 31 - protein, 30 aa, Artificial Sequence
  Human immunodeficiency virus, artificially synthesized
YGRKKRRQRRRGGSIYRRGARRWRKLYRAN

SEQ ID NO: 32 - protein, 26 aa, Artificial Sequence
  Human immunodeficiency virus, artificially synthesized
YGRKKRRQRRRCCRLYRKRIWRSAGR

SEQ ID NO: 33 - protein, 30 aa, Artificial Sequence
  Human immunodeficiency virus, artificially synthesized
YGRKKRRQRRRCCRLRYRNKRIWRSAYAGR

SEQ ID NO: 34 - protein, 26 aa, Artificial Sequence
  Human immunodeficiency virus, artificially synthesized
YGRKKRRQRRRGGRLYRKRIWRSAGR

SEQ ID NO: 35 - protein, 30 aa, Artificial Sequence
  Human immunodeficiency virus, artificially synthesized
YGRKKRRQRRRGGRLRYRNKRIWRSAYAGR